(19) United States
(12) Patent Application Publication
(10) Pub. No.: US 2001/0041349 A1
(43) Pub. Date: Nov. 15, 2001

(54) Protein expression system arrays and use in biological screening

(76) Inventors: Andrew Patron, San Diego, CA (US); Reyad Sawafta, Greensboro, NC (US); Bin Zhou, Edmond, OK (US)

Correspondence Address: Cynthia B. Rothschild, Kilpatrick Stockton LLP, 1001 West Fourth Street, Winston-Salem, NC, 27101, US

(21) Appl. No.: 09/836,746

(22) Filed: Apr. 17, 2001

Related U.S. Application Data
(60) Provisional application No. 60/197,692, filed on Apr. 17, 2000.

(51) Int. Cl. G01N 33/53; G01N 33/537; G01N 33/543; C12Q 1/68; G06F 19/00; G01N 33/48; G01N 33/50

(52) U.S. Cl. 435/7.92; 435/6; 702/19

(57) ABSTRACT

The present invention relates to the generation of an array of protein expression systems for parallel in vitro screening of small molecule libraries, protein or peptide libraries, or other protein-binding components. In an aspect, the invention provides a spatially defined array of protein expression systems comprising: (a) a substrate; (b) a binding surface which covers some or all of the substrate surface; and (c) a plurality of discrete protein expression systems arranged in discrete positions on portions of said substrate covered by said binding surface. Also described are method of using the array for the rapid identification of compounds of able to interact with proteins expressed by any given array.

### CROSS REFERENCE TO RELATED APPLICATIONS

This application claims priority to provisional patent application 60/197,692 filed Apr. 17, 2000.

### FIELD OF THE INVENTION

The present invention relates to the generation of an array of protein expression systems and high-throughput screening of proteins expressed from such arrays.

### BACKGROUND OF THE INVENTION

A variety of protein expression systems have been used over the years as a tool in biochemical research. These expression systems include, but are not limited to, genetically engineered cell lines that over-express a protein of interest (e.g. receptor, antibody or enzyme) modified bacteria, and phage display libraries of multiple proteins. Thus, proteins prepared through these approaches can be isolated and either screened in solution or attached to a solid support for screening against a target of interest such as other proteins, receptor ligands, small molecules, and the like. Recently, a number of researchers have focused their efforts on the formation of arrays of proteins similar in concept to the nucleotide biochips currently being marketed. For example, WO 00/04389 and WO 00/04382 describe microarrays of proteins and protein-capture agents formed on a substrate having an organic thinfilm and a plurality of patches of proteins, or protein-capture agents. Also, WO 99/40434 describes a method of identifying antigen/antibody interactions using antibody arrays and identifying the antibody to which an antigen binds.

While arrays of proteins, and protein-capture agents provide a method of analysis distinct from nucleotide biochips, the preparation of such arrays requires purification of the proteins used to generate the array. Additionally, detection of a binding or catalytic event at a specific location requires either knowing the identification of the applied protein, or isolating the protein applied at that location of the array and determining its identity. Also, attachment of proteins to an array may not necessarily resemble the physiological conditions required for folding of the protein.

What is needed is a means to identify protein binding events wherein the protein is presented to the binding agent or substrate in its physiological state. Additionally, it would be preferable to have the protein presented in a manner that allows for efficient isolation and identification of the proteins for which binding or catalytic events are detected. Finally, the system should enable rapid analysis of the proteins by coupling of the arrays to detection systems that allow for the rapid, high-throughput analysis of chemical or biological samples.

### SUMMARY

The present invention describes the use of organized arrays of protein expression systems for rapid screening of the ability of compounds of interest to interact with a plurality of proteins and peptides expressed from the array. In one aspect, the present invention provides a spatially defined array of protein expression systems comprising: (a) a substrate; and (b) a plurality of discrete protein expression systems located at discrete positions on portions of the substrate. In an embodiment, the array comprises a binding surface which covers some or all of the substrate surface, wherein the protein expression systems are located at discrete positions on portions of the substrate covered by the binding surface.

The present invention also comprises a method for rapid screening of compounds for the ability of the compound or components therein to bind to proteins. Thus, in another aspect, the present invention comprises a method for screening a plurality of proteins for their ability to interact with a component of a sample comprising the steps of: (a) generating a protein expression array, wherein the array comprises: (i) a substrate; (ii) a binding surface which covers some or all of the substrate surface; and (iii) a plurality of discrete protein expression systems located at discrete positions on portions of the substrate covered by the binding surface; and (b) detecting either directly or indirectly the interaction of the component with proteins expressed from specific sites on the protein expression array.

The method also relates to detection of chemical and biological components immobilized in a biochip format. Thus, in one aspect, the invention comprises detection of chemical or biological components immobilized on a solid phase by multidimensional spectroscopy (MDS) utilizing ion mobility and time of flight mass spectroscopy comprising the steps of: (a) recovering at least a portion of a chemical or biological mixture immobilized on a solid substrate as an electrospray; (b) directing the electrospray to an ion mobility chamber which separates the constituents of the mixture based on size, ionic charge, and shape; and (c) analyzing the resultant spray which emerges from the ion chamber by time-of-flight spectroscopy for a component of interest. In an embodiment, the immobilized components are arranged as an array.

In yet another aspect, the invention comprises computer readable media comprising software code for performing the methods of the invention.

The foregoing focuses on the more important features of the invention in order that the detailed description which follows may be better understood and the present contribution to the art better appreciated. There are additional features of the invention which will be described hereinafter and which will form the specification and claims appended hereto. It is to be understood that the invention is not limited in its application to the details set forth in the following description and drawings. The invention is capable of other embodiments and of being practiced or carried out in various ways.

From the foregoing summary, it is apparent that an object of the present invention is to provide a system comprising arrays of protein expression systems suitable for the rapid screening of new compounds such as potential receptor ligands, small molecules, and the like. It is also apparent that an object of the present invention is to provide a method for the rapid screening of collections of proteins, small molecules and other compounds of interest to interact with a plurality of proteins. Another object of the present invention is provide methods for the rapid screening of biochips comprising chemical or biological components. These, together with other objects of the present invention, along with the various features of novelty which characterize the invention, are pointed out with particularity in the claimed invention with description and drawings herein.

### BRIEF DESCRIPTION OF THE DRAWINGS

FIG. 1 shows a schematic representation of an aspect of an embodiment of the method of the present invention.

FIG. 2 shows an aspect of an embodiment of the array of the present invention with a substrate comprising discrete locations having a binding surface and attached phage comprising an expression system wherein panel A shows a phage binding to the binding surface by antibody to the phage; panel B shows a phage binding to the binding surface by an antibody to an affinity tag on the recombinant protein; and panel C shows a phage binding to the binding surface by an poly-his affinity tag interacting with a metal-coated binding surface.

FIG. 3 shows an aspect of an embodiment of the array of the present invention comprising methods of sequestering proteins produced by a protein expression array of the present invention, wherein panel A shows host cells expressing a soluble protein (bottom panel) and transfer of the expressed protein to a second array (top panel); and panel B shows host cells expressing a soluble protein engineered to include an affinity tag (bottom panel) and transfer of the expressed protein to a second array (top panel); and panel C shows host cells expressing a membrane-bound protein.

FIG. 4 shows an aspect of an embodiment of the array of the present invention comprising measuring protein expressed as an array using multi-dimensional spectroscopy (MDS).

### DETAILED DESCRIPTION OF THE INVENTION

The present invention describes the use of organized arrays of protein expression systems for rapid identification of compounds having the ability to interact with the proteins expressed by any given array. An approach that utilizes protein expression systems in a high throughput mode as a unique and effective method for screening is described. Applications include screening of small molecule libraries, protein or peptide libraries, a plurality of known single compounds, or other compounds of interest. By using protein expression arrays, the expression system which produces a product that interacts with a component of interest is easily isolated. This has the advantage of not only providing data showing an interaction between the compound of interest and the expressed protein, but of also providing the protein sequence information and a rapid means of replication within each location of the array.

Thus, in one aspect, the present invention provides a spatially defined array of protein expression systems comprising: (a) a substrate; (b) a binding surface which covers some or all of the substrate surface; and (c) a plurality of protein expression systems located at discrete positions on portions of the substrate covered by the binding surface.

Preferably, the expression systems produce recombinant proteins. In an embodiment, proteins produced by the expression systems are immobilized. Immobilization of the proteins produced by the expression systems may comprise immobilization of the expression systems in the array. Alternatively, immobilization of the proteins produced by the expression systems may comprise a specific interaction of the expressed proteins with the binding surface of the array. Thus, in an embodiment, the expressed proteins comprise an affinity tag which can interact with the binding surface of the array. In another embodiment, the expressed proteins comprise an epitope which can interact with the binding surface of the array. In yet another embodiment, immobilization of the proteins produced by the expression systems comprises binding of the expressed protein to a second array.

The expression systems used to make up the array will vary depending on the types of compounds that are to be screened against the array. For example, the invention contemplates that each distinct location comprising a binding surface may comprise one protein expression system. Alternatively, each distinct location comprising a binding surface may comprise a plurality of expression systems. In a embodiment, each expression system of an array expresses a discrete protein or peptide. In another embodiment, at least some of the expression systems comprising an array express peptides and protein fragments comprising the same protein. In another embodiment, at least some of the expression systems comprising an array express proteins which are related. Preferably, the proteins are related functionally. Also preferably, the proteins are related structurally.

In an embodiment, at least some of the proteins expressed by the protein expression systems immobilized on the array are members of the same family. More preferably, the protein family comprises growth factor receptors, hormone receptors, neurotransmitter receptors, catecholamine receptors, amino acid derivative receptors, cytokine receptors, extracellular matrix receptors, antibodies, lectins, cytokines, serpins, proteinases, kinases, phosphatases, ras-like GTPases, hydrolases, steroid hormone receptors, insulin receptor and insulin receptor substrates, transcription factors, DNA binding proteins, zinc finger proteins, leucine-zipper proteins, homeodomain proteins, intracellular signal transduction modulators and effectors, apoptosis-related factors, DNA synthesis factors, DNA repair factors, DNA recombination factors, cell-surface antigens, Hepatitis C virus (HCV) proteases, HIC proteases, viral integrases, or proteins from pathogenic bacteria.

Preferably, the expression systems comprise at least 10 discrete locations comprising protein expression systems on the array. More preferably, the expression systems comprise at least 10² discrete locations comprising protein expression systems on one array. Even more preferably, the expression systems comprise at least 10³ discrete locations comprising protein expression systems on one array. Even more preferably, the expression systems comprise at least 10⁴ discrete locations comprising protein expression systems on one array.

Preferably, the array of the present invention comprises between 10 to 10⁴ discrete expression systems on one array. More preferably, the array of the present invention comprises between 10² to 10⁴ discrete expression systems on one array. More preferably, the array of the present invention comprises between 10³ to 10⁴ discrete expression systems on one array.

In an embodiment, the binding surface comprises a compound which interacts with the expression system. More preferably, the binding surface comprises a compound that immobilizes the expression system on the array. Preferably, the binding surface comprises an antibody to the protein expression system. The binding surface may also comprise a hydrogel. Alternatively, the binding surface may comprise a membrane. In yet another embodiment, the binding surface comprises at least one functional group that binds to the substrate and at least one functional group that binds to the protein expression system.

In another embodiment, the binding surface comprises a compound which binds the proteins expressed by the expression systems. Preferably, the binding surface comprises an antibody which binds to an epitope present on the expressed proteins. In yet another embodiment, the binding surface comprises at least one layer of coating material. Preferably, the coating comprises a metal film which recognizes an affinity tag present on the expressed proteins.

In an embodiment, the substrate is selected from the group consisting of silicon, silicon dioxide, alumina, glass, titania, nylon, polypropylene, polyethylene, polystyrene, and acrylamide.

In an embodiment, the array of the present invention comprise a micromachined device. In another embodiment, the array of the present invention comprises a biosensor.

The present invention comprises a method for rapid screening of compounds for the ability of the compound or components therein to bind to proteins. Thus, in one aspect, the present invention comprises a method for screening a plurality of proteins for their ability to interact with a component of a sample comprising the steps of: (a) generating a protein expression array, wherein the array comprises: (i) a substrate; (ii) a binding surface which covers some or all of the substrate surface; and (iii) a plurality of protein expression systems located at discrete positions on portions of the substrate covered by the binding surface; and (b) detecting either directly or indirectly the interaction of the component with proteins expressed from specific sites on the protein expression array.

In an embodiment, the method includes detecting the interaction of components at a particular site on the expression array. In another embodiment, the method comprises transferring the expressed proteins to known locations in a second array and detecting the interaction of components with the second array. Preferably, the method includes characterization of binding of the components to proteins expressed from protein expression systems located at specific positions on the array. Also preferably, the method includes characterization of an alteration in the activity of proteins expressed from protein expression systems located at specific positions on the array. Also preferably, the method comprises characterization of DNA isolated from the expression system for which the interaction is detected.

In an embodiment, the component tested for interaction with the proteins expressed by the protein expression systems of the array comprises a protein or peptide. In another embodiment, the component tested for interaction with the proteins expressed by the protein expression systems of the array comprises a small molecule. In another embodiment, the component tested for interaction with the proteins expressed by the protein expression systems of the array comprises a proprotein. In yet another embodiment, the component tested for interaction with the proteins expressed by the protein expression systems of the array comprises a receptor ligand. Preferably, the ligand is selected from the group consisting of peptides, peptide mimetics, antibodies, natural product extracts, and mixtures of the above.

There are many different types of detection systems suitable for measuring the interaction of components of interest with proteins expressed from the array. In an embodiment, the interaction of said component of a sample with said expression array is measured by multi-dimensional spectroscopy (MDS) utilizing ion mobility and time of flight mass spectroscopy for the detection of biological or chemical products formed as the result of the interaction of components of interest with proteins expressed from specific sites on the protein expression array. Preferably, the method includes the steps of: (a) recovering at least a portion of the biological or chemical products formed as the result of the interaction of components of interest with proteins expressed from specific sites on the protein expression array as an electrospray; (b) directing the electrospray to an ion mobility chamber which separates the constituents of the mixture by size, ionic charge, and shape; and (c) analyzing the resultant spray which emerges from the ion chamber by time-of-flight spectroscopy. In another embodiment, the interaction of the components of a sample with proteins expressed by the expression array is measured by collision induced dissociation (CID).

The method also relates to the general use of multidimensional spectroscopy to the detection of chemical and biological components immobilized in a biochip format. Thus, in one aspect, the invention comprises detection of chemical or biological components immobilized on a solid phase by multidimensional spectroscopy (MDS) utilizing ion mobility and time of flight mass spectroscopy comprising the steps of: (a) recovering at least a portion of a chemical or biological mixture immobilized on a solid substrate as an electrospray; (b) directing the electrospray to an ion mobility chamber which separates the constituents of the mixture based on size, ionic charge, and shape; and (c) analyzing the separated constituents which emerge from the ion chamber by time-of-flight spectroscopy for a component of interest. In an embodiment, the immobilized components are arranged as an array. Preferably, the array comprises a micro-chip format. Even more preferably, the array comprises an array of protein expression systems or products thereof

In yet another aspect, the invention comprises computer readable media comprising software code for performing the methods of the invention.

Thus, the present invention utilizes arrays of protein expression systems for high throughput screening of small molecule libraries, protein or peptide libraries, or single compounds for their ability to interact with a plurality of proteins or peptides. The present invention further describes the analysis of the ability of compounds of interest to interact with proteins expressed by protein expression arrays using a biochip format coupled to high-throughput spectroscopic techniques such as multidimensional spectroscopy utilizing ion mobility and time-of-flight mass spectroscopy.

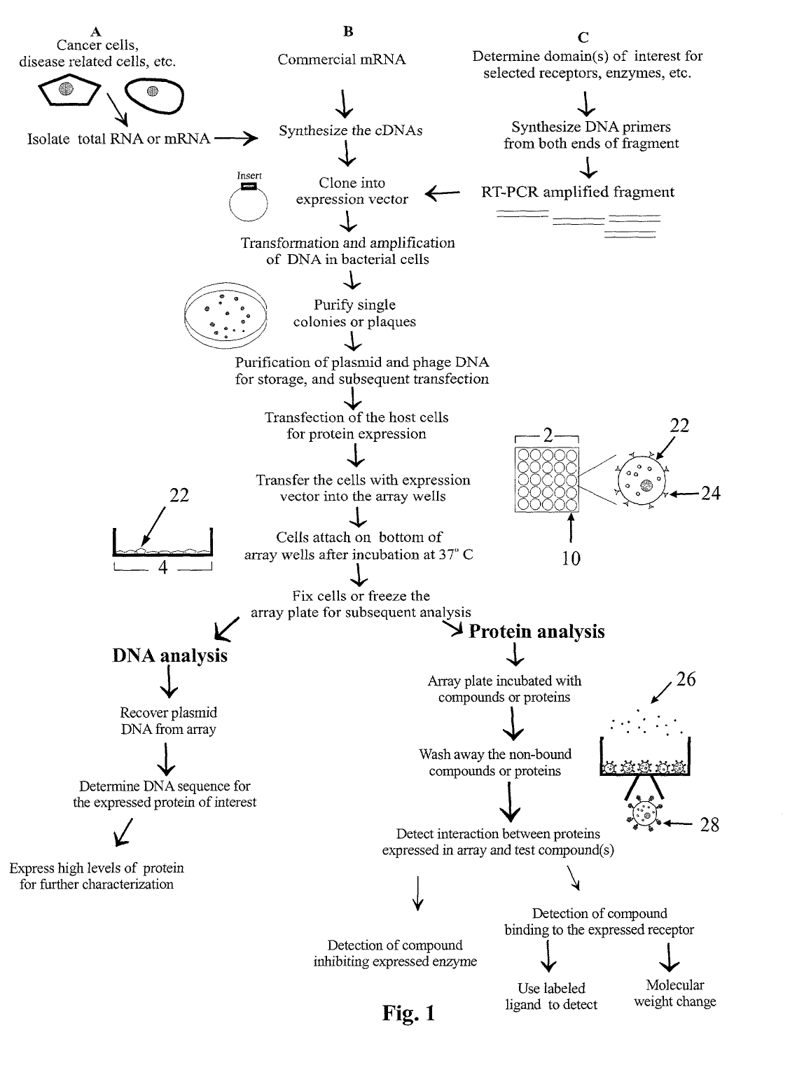

For example, and referring now to FIG. 1, a protein expression library can be created using mRNA, cDNA, or PCR amplified sequences of interest. For example, mRNA may be isolated from a specific cell type (step 1: panel A). Alternatively, pools of mRNA or cDNA libraries from tissue types of interest such as, but not limited to, species-specific libraries, or libraries obtained from specific tumors or organs, may be obtained commercially (step 1: panel B). Alternatively, domains of interest in specific protein types may be identified by computer analysis, and sequences corresponding to such domains synthesized, as for example, by polymerase chain reaction (PCR) amplification using primers which flank the regions of interest (step 1: panel C). Thus, libraries can be tailored to include proteins which are known to be structurally or functionally related, proteins comprising receptor or enzyme subclasses, proteins expressed in different disease states, and the like.

The cDNA (or PCR-amplified DNA) is then subcloned into an expression vector and single clones isolated by colony or plaque purification. After amplification and purification, the recombinant DNA is used to transfect host cells under conditions which provide for efficient protein expression. Individual clones are isolated and the collected recombinants placed in a spatially addressable array. The clones used for any individual array may comprise multiple aliquots of the same recombinant, a collection of related proteins or peptides, or a library of individual recombinants, depending on the array requirements.

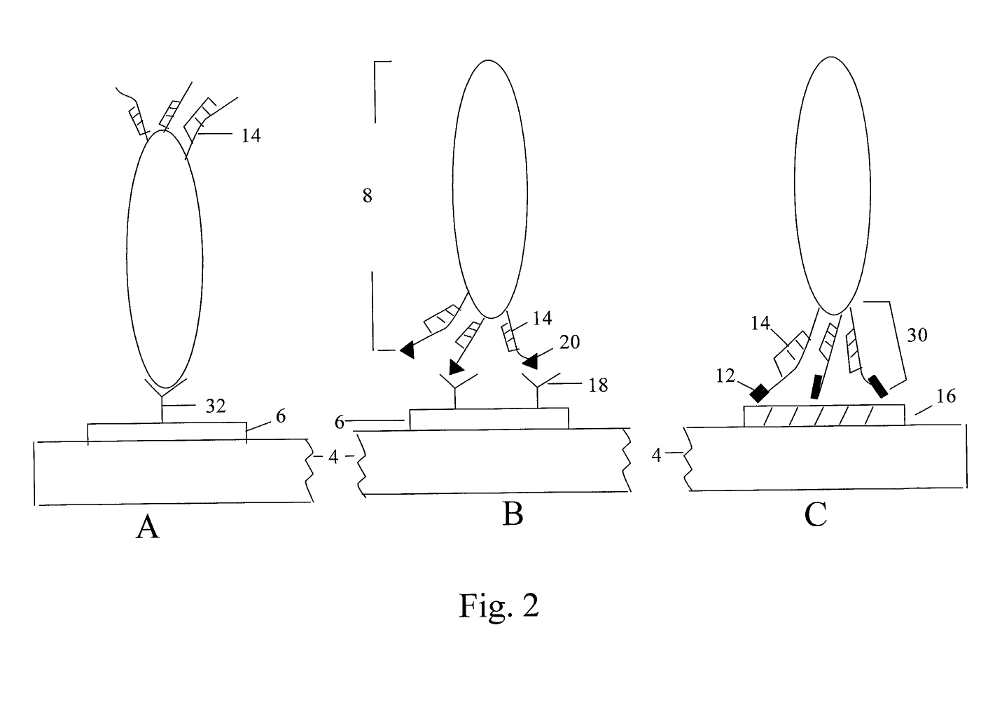

Generally, and referring now to FIGS. 1 and 2, the array 2 of the present invention comprises (a) a substrate 4; (b) a binding surface 6 which covers some or all of the substrate surface; and (c) a plurality of discrete protein expression systems 8 located at discrete positions on portions of the substrate covered by the binding surface. The substrate is generally a base or support on which the array is mounted. For example, the substrate may be a polypropylene microtiter plate, or a glass or plastic rectangular surface (i.e. a chip). On top of the substrate is a binding surface 6 spaced at regular intervals on which the expression systems 8 are located. The binding surface may comprise the wells of a microtiter plate, small recessions on a flat chip-like structure, or patches of membrane arranged in a regular format. The binding surface may also include additional components such as a nutrient layer, a lipid layer, polymers, or a hydrogel. Additionally, the binding surface includes components for immobilization of the proteins expressed by the array. For example, in an embodiment, the binding surface may include a metal coating 16 for binding a poly-histidine (poly-his) affinity tag 12 which may be included in the expressed proteins 14 (FIG. 2C). In another embodiment, the binding surface includes an antibody which recognizes an epitope affinity tag 20 which may be included in the expressed proteins (FIG. 2B).

At this point, the array of protein expression systems may be fixed (e.g. using formaldehyde or other fixing agents known in the art) or frozen (e.g. in 5% dimethlysulfoxide DMSO-media mix) to allow for: (1) immobilization of the recombinant DNA insert/expression vector and (2) assay of expressed proteins (FIG. 1).

As shown in FIG. 1, to assay expressed proteins, the array 2 of cells 22 expressing recombinant protein 24 may be incubated with a compound of interest 26 and the ability of that compound to interact with expressed proteins 24 assayed. In some cases, as for example, where the expressed protein comprises a majority of the protein produced, or where the expressed proteins are bound to the surface of the expression system host cell, expressed proteins can be assayed in situ (i.e. at the array site comprising the expression system). For example, in the embodiment shown in FIG. 1, the recombinant sequence expresses a membrane bound protein 24 which localizes in the membrane of the host cell 22. In another embodiment, the array comprises a phage display library, in which the recombinant protein/peptide 14 comprises part of the extracellular phage filament 30 (FIG. 2). Also, recombinant proteins may be engineered to contain an anchor or membrane binding sequence, thus localizing the expressed sequences to the membrane of the host cell.

In some cases, however, it may be preferable to select for the expressed proteins prior to assay. For example, the proteins expressed by the expression system may include an affinity tag. The affinity tag allows for immobilization of expressed protein as a result of binding of the tag to its binding partner. In an embodiment, recombinant proteins are engineered to include a poly-his affinity tag (e.g. (His)₆). Proteins expressing the poly-histidine tag can be immobilized by binding of the tag to metals, such as zinc, nickel, cobalt, or commercial metal preparations such as TALON, and the like. Alternatively, proteins expressing affinity tags may be immobilized by binding of the affinity tag to protein binding partners such as antibodies and the like. For example, proteins expressing the poly-his tag can also be immobilized by binding to antibodies that recognize poly-his. Thus, the binding surface of the array may include either a metal coating or antibody to poly-his. Alternative affinity tags which can be recognized by antibodies specific for the tag epitope include a nine amino acid epitope from the human c-myc protein; a twelve amino acid epitope from protein-C; hemagglutinin (HA), or FLAG 8.

Thus, in an embodiment, and referring again to FIG. 2, a desired protein expression system is selected and the gene or genes for the proteins of interest incorporated into a phage display library. The phagemid vector may be engineered so that the sequence encoding (His)₆ is inserted adjacent to the M13 gene sequences which allow for expression of the cloned sequence. Thus, recombinant phage can be selected by binding to anti-M13 antibody (panel A) or binding to antibody specific for the poly-his tag (panel B), or by binding of the poly-his tag to a metal impregnated binding surface (panel C).

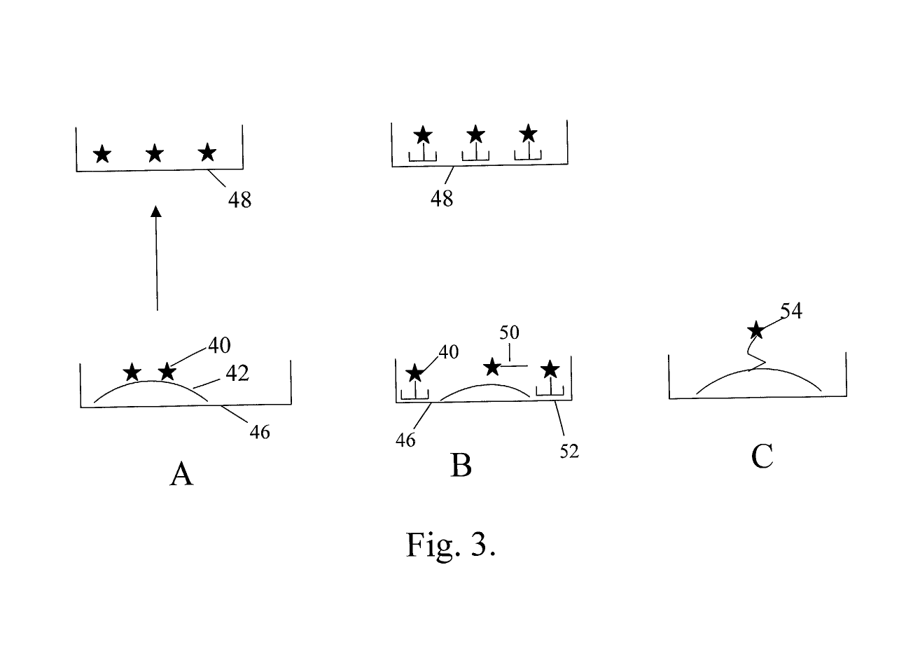

Recombinant proteins may be assayed either in the expression array, or after transfer of the proteins to a second array format. For example, an array of protein expression systems may be distributed in the wells of a microtiter-like array. Referring now to FIG. 3, in the case of soluble protein 40 secreted from cells 42, the presence of the protein may be evaluated directly in the well 46, or after transfer of the secreted components to another well 48 (FIG. 3A, bottom and top panels, respectively). Similarly, where the soluble protein is cytosolic, the cells may be lysed and the recombinant protein measured directly in the well, or after transfer of the secreted components to another well. In either case, detection of expressed protein does not compromise isolation of the plasmid/phagemid DNA from each site of the array. Thus, for the array site which provides an interaction of interest, the recombinant DNA can be isolated and propagated for further characterization.

Alternatively, as shown in FIG. 3B, recombinant proteins 40 expressed with affinity tags 50 may be immobilized by binding of the tag to its binding partner 52. The binding partner may be immobilized in the expression array 46, or the tagged protein can be transferred to a second array 48 comprising a binding surface and substrate. For immobilization in the expression array, sites on the binding surface of the expression array 46 may include a metal (for binding poly-his) or antibody coating (for binding other epitope tags) so that proteins secreted from the expression system (or released upon lysis of the host cells) can be immobilized in the primary array (FIG. 3B, bottom). Alternatively, the binding surface of a secondary array may include a metal or antibody coating to allow immobilization of expressed proteins in the secondary array (FIG. 3B, top).

In another embodiment, recombinant proteins are expressed as membrane bound proteins 54. For example, membrane proteins such as receptors, or ion channels are expressed as membrane bound proteins. In addition, recombinant proteins may be engineered to include secretion signal sequence such as mouse Ig kappa-chain for efficient secretion recombinant proteins with expressed protein transmembrane domain (pSecTag 2; Invitrogen, Carlsbad, Calif.) or the transmembrane domain such as PDGFR (platelet derived growth factor receptor) for protein to display on the cell surface (pDisplay vector; Invitrogen).

The expressed proteins can then be exposed to a plurality of compounds of interest, such as small molecules, peptides, proteins, or potential ligands. For soluble proteins, interaction of the expressed protein with a compound of interest may employ measurement by spectroscopic methods. For example, measurement of a binding event would entail detection of a change in molecular weight or quenching of a fluorescent ligand. Similarly, production of an enzyme product, or loss of a substrate may be detected using methods known in the art.

For expressed proteins which are immobilized in either the primary array of protein expression systems (FIG. 3, lower panels) or in a secondary array (FIG. 3, upper panels), assays employing the solid phase may be employed. For example, a phage display library may be immobilized in an array by binding of a his-tag which has been engineered into the recombinant proteins to a metal binding surface (FIG. 2C). Similarly, membrane bound proteins expressed from host cells may be immobilized in the array by allowing the cells to attach to the binding surface (FIG. 1). The immobilized expression systems may then be incubated with selected compounds of interest (FIG. 1). After incubation with the immobilized systems, any non-binding compounds can be washed away and binding interaction with the various proteins detected by various analytical methods such as, but not limited to, measurement of radiolabeled ligands, internalization of a radiolabeled or fluorescent ligand, enzyme-linked immunoassay (ELISA) and the like.

After detection of a binding interaction, the desired or plasmid DNA (or in the case of a phage display library, the phage itself), can be specifically eluted from the array, transferred to its host organism and re-expressed, providing both additional protein for further studies and the sequence coding for that protein. The process considerably reduces the amount of time needed for the collection of both protein and gene data, allows for rapid reiteration of the process if necessary, and eliminates the need for detailed protein or gene sequence data prior to the assay.

The general principles described above are exemplified in the specific systems described in more detail below.

### Definitions

A “protein” is a polymer of amino acid residues linked together by peptide bonds, and as used herein refers to proteins and polypeptides of any size structure or function. A protein may be naturally occurring, recombinant or synthetic. A protein may include one or more amino acid residues which comprise an unnatural amino acid or an artificial chemical analogue of a naturally occurring amino acid.

A “fragment of a protein” means a protein which is a portion of another protein. Peptides constitute protein fragments. A fragment of a protein will typically constitute 6 amino acids or more, but in some cases may be fewer.

The term “antibody” comprises an immunoglobulin, whether natural or synthetically produced. An antibody may be polyclonal or monoclonal. Polyclonal antibodies are a heterogeneous population of antibody molecules derived from the sera of animals immunized with the antigen of interest. Adjuvants such as Freund's (complete and incomplete), peptides, oil emulsions, lysolecithin, polyols, polyanions and the like may be used to increase the immune response. The antibody may be a member of any immunoglobulin class including: IgG, IgM, IgA, IgD and IgE. Monoclonal antibodies are homogeneous populations of antibodies to a particular antigen, and are generally obtained by any technique which provides for production of antibody by continuous cell lines in culture (see e.g. U.S. Pat. No. 4,873,313).

The term “micromachining” and “microfabrication” refer to techniques used in the generation of microstructures comprising features having sub-millimeter size. Such technologies include, but are not limited to, laser ablation, electrodeposition, physical and chemical vapor deposition, photolithography, wet and dry etching, injection molding and x-ray lithography, electrodeposition and molding.

A “binding surface” comprises a layer applied to the substrate (or to coating on a substrate) which comprises distinct locations on which the protein systems of the array are located. Typically, the binding surface comprises an organic surface, such as polypropylene, or a membrane. A hydrogel, or lipid, or polymer may also comprise the binding surface. The binding surface will preferably comprise exposed functionalities useful in binding expressed proteins to the array. Alternatively, the binding surface may bear functional groups which reduce non-specific binding. Additionally, the binding surface may comprise functionalities designed to enable the use of certain detection techniques.

The present invention also contemplates the use of affinity tags for immobilizing the expression library on the substrate. An “affinity tag” may be a simple chemical group, or may include amino acids, poly-amino acids, or full length proteins which bind to a specific binding partner, such as a metal coating or an antibody. Typical affinity tags include polyhistidine (His₆), human c-myc protein (nine amino acid epitope), protein-C (a twelve amino acid epitope from the heavy chain of human protein-C), and Hemagglutinin (HA).

A protein expression system comprises a biological system which is able to express proteins. An in vivo protein expression system generally comprises a host cell transformed with a recombinant DNA molecule including sequences which are translated into protein products. An in vitro protein expression system generally comprises cellular machinery which enables the translation of MRNA.

A recombinant protein comprises a protein which is derived from a DNA sequence that has been modified in some way.

A “small molecule” comprises a compound or molecular complex, either synthetic, naturally derived, or partially synthetic, composed of carbon, hydrogen, oxygen, and nitrogen, which may also contain other elements, and which preferably has a molecular weight of less than 5,000. More preferably, a small molecule has a molecular weight of between 100 and 1,500.

A “peptide mimetic” comprises a molecule which embodies the character of a peptide in the inclusion of side chains and amide (peptide) bonds typical of a peptide, with one or more chemical modifications to the peptide structure including the amide bonds and/or the side chains. An example of a peptide mimetic would include peptides where the groups —CH₂CH(OH)— or —CH₂—CH₂— are substituted for one or more —NH—C(O)— peptide bonds.

A biochip comprises a substrate having a surface to which one or more arrays of probes is attached. The substrate can be, merely by way of example, silicon or glass and can have the thickness of a glass microscope slide or a glass cover slip. Substrates that are transparent to light are useful when the method of performing an assay on the chip involves optical detection.

Microchips comprise integrated circuit elements, electrooptics, excitation/detection systems and nucleic acid based receptor probes in a self-contained and integrated microdevice. A basic microchip, for example, may include: (1) an excitation light source; (2) a bioreceptor probe; (3) a sampling element; (4) a detector; and (5) a signal amplification/treatment system.

### Expression Systems

There are many different types of protein expression systems. Several cell-free protein systems can be used for in vitro transcription and translation of mRNA isolated from various sources. These in vitro translation systems simplify the transcription of cDNA or PCR-amplified DNA sequences cloned in vectors such as, but not limited to, plasmids, providing a powerful tool for identifying and characterizing polypeptides.

Rabbit reticulocyte lysate and wheat germ extract both provide a reliable, convenient, and easy to use systems to initiate translation and produce full size polypeptide products. Reticulocyte lysate is often favored for translation of larger mRNA species, and is generally recommended when microsomal membranes are to be added for co-ranslational processing of translation products. Wheat germ extract readily translates certain RNA preparations, such as those containing low concentrations of dsRNA or oxidized thiols, which are inhibitory to reticulocyte lysate. This system supports the translation in vitro of a wide variety of viral, prokaryotic, and eukaryotic mRNAs into protein. Translation reactions in vitro may be directed by either mRNA isolated in vivo or by RNA templates transcribed in vitro from commercial vectors (e.g. pGEM vector used in Riboprobe System; Promega, Madison, Wis.).

DNA sequences cloned in plasmid vectors also may be expressed directly using E. coli S30 coupled transcription translation system (Promega, Madison, Wis.). The template DNA to be expressed must contain prokaryotic promoter sequences and ribosome binding sites. Two types of S30 systems are available. The standard systems allow for the expression of cloned DNA fragments present in super-coiled plasmid vectors under control of an Escherichia coli promoter. The second type of S30 system is generated from an E. coli strain that allows either plasmid DNA or linear DNA to be transcribed and subsequently translated. E. coli-based protein expression is generally the method of choice for soluble proteins that do not require extensive post-translational modifications for activity. For E. coli expression, DNA sequences are ligated into expression vector (usually under an inducible promoter) and introduced into the appropriate competent E. coli strain (e.g. XL-1 blue, BL21, SG13009) by calcium-dependent transformation or electroporation. Transformed E. coli cells are plated and individual colonies transferred into 96-well microtiter arrays or similar array-like formats.

Choosing the right eukaryotic system for the expression of a eukaryotic gene can be particularly important in obtaining biologically active recombinant protein. For example, Saccharomyces cerevisiae allows for core glycosylation and lipid modifications of proteins. Alternatively, baculovirus expression systems provide an environment where an over-expressed recombinant protein has proper folding, disulfide bond formation, and oligomerization. Additionally, the baculovirus system is capable of performing most of the post-translational modifications such as N— and 0— linked glycosylation, phosphorylation, amidation and, carboxymethylation. For example, insect cells are increasingly used for production of recombinant proteins using baculovirus. In most cases, posttranslational processing of eukaryotic proteins in insect cells is similar to protein processing in mammalian cells. A baculovirus commonly used to express foreign proteins is Autographa californica nuclear polyhedrosis virus (AcMNPV) (see e.g. Luckow, BioTechnology 6:47-55 (1991)). For example, replacement of polyhedrin gene sequences with an inserted foreign sequence enables expression of the inserted gene by the polyhedrin promoter. The polyhedrin protein, while essential for propagation of the virus in its natural habitat, is not required for propagation of the virus in cell culture, and thus, can be replaced with a foreign sequence.

Because the AcMNPV genome is fairly large, recombinant baculovirus expression vectors may employ recombination between a transfer vector comprising insert DNA and the viral genome. For example, in the pBacPAK system (Clontech, Palo Alto, Calif.) a target gene is cloned into a polyhedrin locus which is contained in a relatively small (<10 kb) transfer vector. The polyhedrin locus in the transfer vector has the coding sequence deleted and replaced with a multiple cloning site (MCS) for insertion of a target gene between the polyhedrin promoter and polyadenylation signals. In a second step, the transfer vector (which is unable to replicate on its own in insect cells) and a viral genomic DNA are co-transfected into insect cells. Double recombination between viral sequences in the transfer vector and the corresponding sequences in the viral DNA transfers the target gene to the viral genome to generate a viral expression vector.

Libraries may also be propagated using phage display. Phage display is a technique which allows the expression of a defined specificity on a viable organism (bacteriophage) thereby permitting the identification of that specificity and isolation to be accomplished on an immunosorbent surface. Phage display provides a general selection technique in which a peptide or protein is expressed as a fusion product with a coat protein of a bacteriophage, resulting in display of the fused protein on the exterior surface of the phage virion, while the DNA encoding the fusion protein resides within the virion. In the specific case of M13 phage, a large repertoire of molecules can be expressed on the phage surface (see e.g. U.S Pat. No. 5,969,108; U.S. Pat. No. 5,733,743; U.S. Pat. No. 5,871,907; U.S. Pat. No. 5,858,657; U.S. Pat. No. 5,977,322; WO 90/02809; Barbas, C. F., et al., Proc. Natl. Acad. Sci. USA, 88:7978-82 (1991); Winter G., et al., Annu. Rev. Immunol., 12:433-55 (1994); Marks J. D. et al., J Biol. Chem. 267:16007-16010 (1992); Soderlind, E. et al., Immunol. Rev., 130:109-124 (1992), although there are some constraints on the size of acceptable inserts.

Phage display recombinants expressing a molecule of interest are selected by assays appropriate for the expressed sequence. Generally, phage with inserts are purified by “panning” against a binding partner which recognizes the peptide expressed on the surface of the virion filaments (see e.g. Parmley, S. F., et al., Gene, 73:305-318 (1988); de Bruin, R., et al., Nature Biotechnology, 17:397-399 (April 1999)). Biopanning involves incubating a library of phage-displayed peptides with a plate (or bead) coated with the target, washing away the unbound phage, and eluting the specifically-bound phage. In an alternative approach, the phage can be reacted with the target in solution, followed by affinity capture of the phage-target complex(es) onto a plate or bead that specifically binds the target. The eluted phage is then amplified and taken through additional cycles of biopanning and amplification to successively enrich the pool of phage in favor of the tightest binding sequences. After several (3-4) rounds, the individual clones are characterized by DNA sequencing and ELISA. Phage which bind to the immobilized binding partner are propagated in E. coli to permit sequencing of the inserts (Scott et al. (1990)) or for large-scale production of either soluble, or phage-expressed protein.

The utility of this approach to small molecule screening has recently been demonstrated in a study in which FKBP (FK506 binding protein) was identified as the protein that binds the immunosuppressive drug, FK506. In this study, FK506 was linked to a solid support and used as an affinity column to assay binding of T7 phage libraries (Austin et al., Chem. Biol., 6, 707 (1999)). In a similar approach, the natural target of Ilimaquinone (Snapper et al., Chem. Biol., 6, 639 (1999)) was identified.

### Organization of Expression Systems on the Array

Typically, the arrays comprise centimeter scale, two dimensional arrangements of protein expression systems immobilized on a binding surface on the surface of a substrate. The array itself can range from the standard microtiter plate format (e.g. 24, 48, 96, 384, or 1536 wells), to a small micro array containing hundreds of spots within 1 to several cm².

Thus, in an embodiment, the expression systems comprises at least 2 discrete locations on an array. Preferably, the expression systems comprise at least 10 discrete locations on one array. More preferably, the expression systems comprise at least 10² discrete locations on one array. Even more preferably, the expression systems comprise at least 10³ discrete locations on one array. Even more preferably, the expression systems comprise at least 10⁴ discrete locations on one array.

Similarly, the specific arrangement of expression systems organized on each array may be expected to vary with particular applications. Preferably, the array of the present invention comprises at least 10 discrete expression systems on one array. More preferably, the array of the present invention comprises at least 10² discrete expression systems on one array. More preferably, the array of the present invention comprises at least 10³ discrete expression systems on one array. Even more preferably, the array of the present invention comprises at least 10⁴ discrete expression systems on one array.

The surface area of the substrate covered by each expression system (and associated binding surface) is preferably less than 0.5 cm². More preferably, the area covered by each expression system covers an area ranging from 1 mm² to about 0.1 cm². Even more preferably, the area covered by each expression system covers an area ranging from 1 cm² to about 0.05 cm².

The distances between each expression system vary depending on the layout of the array. For example, in an embodiment, two or more expression systems are arranged in a section of an array comprising a total area of about 1 cm² or less. In a preferred embodiment, 5 or more expression systems are arranged in a section of an array comprising a total area of about 1 cm² or less. Even more preferably, 10 or more expression systems are arranged in a section of an array comprising a total area of about 1 cm² or less.

In an embodiment, each protein expression system expresses a discrete expressed protein or peptide. In another embodiment, at least part of an array expresses a plurality of peptides and protein fragments comprising a single protein. Thus, it is anticipated that an array may comprise multiple locations, each having the same expression system (as for example, where a protein of interest is screened against a library of unknowns). In another embodiment, at least part of an array expresses a plurality of related proteins. Preferably, the proteins are related functionally. Also preferably, the proteins are related structurally.

For example, the proteins expressed by the protein expression systems immobilized on the array may be members of the same family. In an embodiment, the families include, but are not limited to, families of growth factor receptors, hormone receptors, neurotransmitter receptors, catecholamine receptors, amino acid derivative receptors, cytokine receptors, extracellular matrix receptors, antibodies, lectins, cytokines, serpins, proteinases, kinases, phosphatases, ras-like GTPases, hydrolases, steroid hormone receptors, transcription factors, DNA binding proteins, zinc finger proteins, leucine-zipper proteins, homeodomain proteins, intracellular signal transduction modulators and effectors, apoptosis-related factors, DNA synthesis factors, DNA repair factors, DNA recombination factors, cell-surface antigens, Hepatitis C virus (HCV) proteases, HIC proteases, viral integrases, and proteins from pathogenic bacteria. In an embodiment, the proteins expressed by the array include a family comprising antigens. In an embodiment, the proteins expressed by the array include a family comprising antibodies.

### Array Format

The method of attachment will vary with the substrate and protein expression system selected. For example, in the case of a phage display library, the method of attachment can involve either the direct attachment of the phage as for example, by anti-M13 antibodies, or by attachment via the recombinant protein as for example via antibodies to an epitope-tag incorporated in the recombinant sequence, or by binding of a his-tag incorporated in the recombinant sequence to a metal coating on the binding surface.

Generally, the substrate comprises a support for the array, and thus, may by made of almost any material. Thus, the substrate may be organic, inorganic, biological or synthetic. In an embodiment, the substrate comprises a polypropylene microtiter plate. In another embodiment, the substrate comprises a rectangular chip-like format. In yet another embodiment, the substrate may be a glass microscope slide or similar support. In an embodiment the substrate comprises a nutrient layer.

Numerous materials may be used for the substrate including, but not limited to, silicon, silicon dioxide, alumina, glass, titania, nylon, polycarbonate, polypropylene (and derivatives thereof), polyethylene (and derivatives thereof), polystyrene (and derivatives thereof), and polyacrylamide (and derivatives thereof). Other substrate materials include poly(tetra)fluoroethylene, polyvinylidenedifluoride, polymethylmethacrylate, polyvinylethylene, polyethyleneimine, polyvinylphenol, polymethacrylimide, polyhydroxyethylmethacrylate (HEMA). In an embodiment, the expression systems attach directly to the substrate.

The binding surface comprises the surface on which each of the expression systems is immobilized. Binding surfaces comprise materials suitable for immobilization of expression arrays. Suitable binding surfaces include membranes, such as nitrocellulose membranes, polyvinylidenedifluoride (PVDF) membranes, and the like. Alternatively, the binding surface may comprise a hydrogel. For example, dextran may serve as a suitable hydrogel. Alternatively, the binding surface comprises an organic thin film such as lipids, charged peptides (e.g. polylysine or poly-arginine), or a neutral amino acid (e.g. polyglycine).

The binding surface may include a coating. The coating may be formed on, or applied to, the binding surface. For example, in an embodiment, the coating is a metal film. Metals which may be used for coating include, but are not limited to, gold, platinum, silver, copper, zinc, nickel, cobalt. Additionally, commercial metal-like substances may be employed such as TALON metal affinity resin and the like. Coatings may be applied by electron-beam evaporation or physical/chemical vapor deposition. In another embodiment, coatings comprise functional groups that react with the substrate, including, but not limited to silicon oxide, tantalum oxide, silicon nitride, alumina, glass, and the like. The coating may cover the entire substrate, or may be limited to regions comprising an associated binding surface.

The coating may comprise a component to reduce non-specific binding. Or, the coating may comprise an antibody. For example, antibodies which recognize epitope tags engineered into the recombinant proteins may be employed. Alternatively, recombinants may be generated comprising a poly-histidine affinity tag. In this case, an anti-histidine antibody chemically linked to the substrate provides a binding surface for immobilization of the expression systems. For example, in one embodiment, a polypropylene substrate is coated with a compound, such as bovine serum albumin, to reduce non-specific binding, and then a binding surface comprising dextran functionally linked to a receptor which recognizes M13 epitopes is added to distinct locations on the coating such that phage expressing recombinant proteins will be bound. In another embodiment, the coating comprises a nutrient layer.

A variety of techniques known in the art may be used to generate an array of binding surfaces. For example, patches of an organic thinfilm may be generated by microstamping (U.S. Pat. Nos. 5,512,131 and 5,731,152), microfluidics printing, inkjet printers, or manually with multichannel pipets.

The binding surface may also comprise a compound which has the ability to interact with both the substrate and the expression system. For example, functionalities enabling interaction with the substrate may include hydrocarbons having functional groups (e.g. —O—, —CONH—, CONHCO—, —NH—, —CO—, —S—, —SO—), which may interact with functional groups on the substrate. Functionalities enabling interaction with the expression system comprise antibodies, antigens, receptor ligands, compounds comprising binding sites for affinity tags, and the like.

### Proteomics

The protein expression array of the present invention can have many applications such as, but not limited to, proteomics. For example, the array can express proteins or fractions of proteins from growth factor receptors, insulin receptor and insulin receptor substrates, nuclear orphan receptors, hormone receptors, neurotransmitter receptors, cytokine receptors, extracellular matrix receptors, antibodies, lectins, cytokines, proteases, kinases, phosphatases, ras- like GTPases, hydrolases, steroid hormone receptors, transcription factors, DNA binding proteins, leucine-zipper proteins, homeodomain proteins, intracellular signal transduction modulators and effectors, apoptosis-related factors, DNA synthesis factors, DNA repair factors, DNA recombination factors, cell-surface antigens, hepatitis C virus (HCV), proteases, HIV proteases, viral integrases or proteins from pathogenic bacteria.

Also, an array may comprise selected peptide domains from a specific protein. In this embodiment, an array is used to map specific regions of the protein for the ability to interact directly or indirectly with compounds of interest.

The arrays of the present invention are therefore useful for epitope mapping, the study of protein-protein interaction, binding of drug candidate to a plurality of proteins, drug-drug interaction (for example competition binding studies of two drug candidates), binding of a plurality of drug candidates to a single or several proteins, diagnostics, or antigen mapping.

### Methods for Assaying Interactions of Compounds of Interest with Proteins Expressed by the Array

Use of the array of the invention optionally comprises simultaneous assay of each expression loci. For arrays comprising three dimensional well formats, multichannel pipets may be used. For some applications, the entire array may be submersed in a flow chamber. In an embodiment, a flow chamber comprises approximately 10-20 μl fluid per 25 mm² surface area. Regardless of the exact format, assays should comprise physiological pH and ionic strength to preserve correct protein folding and activity.

For measurement of binding interactions, a step comprising blocking of non-specific binding may be employed. For example, for antibody antigen reaction, the array may be exposed to a blocking solution (such as bovine serum albumin in a physiological buffer) to prevent nonspecific protein interactions. For an antigen-expressing array, antibody is then added, and the amount of antibody bound to each expression system detected. For an antibody expressing array, an antigens are added, and the amount of antigen bound to each expression system detected.

### Detection Systems

The use of expression system arrays and microchip-based separation devices for the rapid analysis of large numbers of samples will introduce a quantum jump in the speed with which samples can be characterized and analyzed. The present invention thus comprises coupling high throughput detection systems to protein expression arrays and the products thereof The ability to couple a biochip array to a system comprising high-speed parallel processing of samples comprises a significant reduction in analysis time. Also, the ability to perform high-throughput sequential and/or parallel separation and detection of sample components using micro-chip arrays significantly reduces the volume of wet chemistry reagents required, thereby reducing the cost of analysis.

There are many different types of detection systems suitable to assay the protein expression arrays of the present invention. Such systems include, but are not limited to, fluorescence, measurement of electronic effects upon exposure to a compound or analyte, luminescence, ultraviolet visible light, and laser induced fluorescence (LIF) detection methods, collision induced dissociation (CID), mass spectroscopy (MS), CCD cameras, electron and three dimensional microscopy. Other techniques are known to those of skill in the art. For example, analyses of combinatorial arrays and biochip formats have been conducted using LIF techniques that are relatively sensitive (e.g. S. Ideue et al., Chemical Physics Letters, 337:79-84, 2000).

One detection system of particular interest is time-of-flight mass spectrometry (TOF-MS). Using parallel sampling techniques, time-of-flight mass spectrometry may be used for the detailed characterization of hundreds of molecules in a sample mixture at each discreet location within the array. Time-of-flight mass spectrometry based systems enable extremely rapid analysis (microseconds to milliseconds instead of seconds for scanning MS devises) high levels of selectivity compared to other techniques with good sensitivity (better than one part per million, as opposed to one part per ten thousand for scanning MS), As a mass spectroscopic technique, time-of-flight mass spectrometry provides molecular weight and structural information for identification of unknown samples.

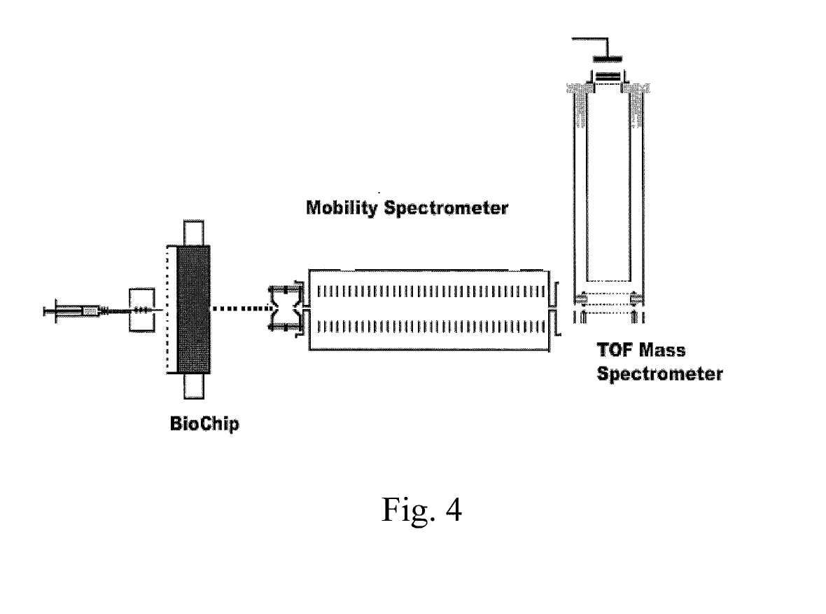

Additional levels of sensitivity are added by coupling time-of-flight mass spectrometry to another separation system. Thus, in an embodiment, and referring now to FIG. 4, the present invention comprises using ion mobility in combination with time-of-flight mass spectrometry for the analysis of micro-arrays. The combination of ion mobility and time-of-flight mass spectrometry is referred to as multi-dimensional spectroscopy (MDS). Ions are electro-sprayed into the front of the MDS device. Electrospray is a method for ionizing relatively large molecules and having them form a gas phase. The solution containing the sample is sprayed at high voltage, forming charged droplets. These droplets evaporate, leaving the sample's ionized molecules in the gas phase. These ions continue into the ion mobility chamber where the ions travel under the influence of a uniform electric field through a buffer gas. The principle underlying ion mobility separation techniques is that compact ions undergo fewer collisions than ions having extended shapes and thus, have increased mobility. As the separated components (comprising ions/molecules of different mobility) exit the drift tube, they are pulsed into a time-of-flight mass spectrometer.

The instrument is designed so that the mobility and mass of individual components in a mixture is recorded in a single experimental sequence. Flight times of ions in the mass spectrometer are recorded within individual drift time windows. By coupling separation due to ionic mobility with time-of-flight mass spectrometry, an extra degree of freedom is introduced into the detection system. The extra degree of freedom results in an increase in sensitivity as components are separated on the basis of charge, shape and mass. Thus, MDS allows for detection of differences of as little as one unit mass or one unit ionic charge in the products at each site of an array. In contrast, conventional ion mobility/mass spectrophotometry methods that utilize mass filters (selecting for ions based on mass/charge (m/z) ratio) discard all ions except those having a selected m/z range, thus narrowing the analysis. MDS allows distributions of ions to be separated by differences in mobility before they are dispersed by differences in their m/z ratios, thereby making it possible to measure m/z ratios for all components of a mixture of mobility-separated ions simultaneously.

Also, because the density of gas is much lower than condensed phase of a compound, gas-phase separations are rapid, usually requiring milliseconds. The timescale for the separation phase of an ion mobility experiment, therefore, is intermediate between the microsecond timescale required for high-throughput mass spectrometry (such as time of flight mass spectroscopy) and the second to minute time scale of condensed phase separations. This time differential allows a three-dimensional separation to be carried out in a nested fashion. That is, time of flight distributions can be recorded within individual drift time windows, allowing a two-dimensional dispersion of ion species as they exit the ion mobility column.

Thus, the technology for gas-phase separation provides the ability to detect ions from a variety of condensed phase separations, using a multidimensional approach such as but not limited to array position, mobility and m/z dispersion. This allows mixtures of tremendous complexity to be examined in a single measurement. The mobility dimension of the MDS is sensitive to structural variations of isomers that cannot be resolved by mass spectrometry alone.

A preferred method to couple the microchip based separation device to a detection system is the use of an electrospray source that can be interfaced between the output of the separation channel on the chip and a detection system based on either an atmospheric pressure ionization or an evacuated TOF-MS. The separation method utilized with TOF-MS (and other detections systems described below) may comprise electrophoresis, preferably utilizing electrochromatography as a means to separate ions based on both adsorption as well as migration. Electrospray and capillary electrophoresis both require high voltages, so the system should decouple the fields necessary for good separation efficiency and electrospray. An external sprayer coupled to the microchip by a liquid junction using readily available fused silica tubing allows for a very simple chip design that can be made of but not limited to glass or polymer. This approach minimizes the dead volume of the system and also allows for adding proper solvents and additives for good electrospray behavior. FIG. 5, shows a possible layout for such an interface.

In an embodiment, an electrospray device provides a reproducible controllable, robust means of producing nanoelectrospray of liquid sample from a silicon microchip (e.g. Cornell University Nanofabrication Facility, http://www.cnf.cornell.edu/). Thus, an electrospray device may be fabricated from a monolithic silicon substrate using reactive ion-etching and other standard semiconductor techniques. The electrospray device for MDS analysis of the biochips of the present invention produces a stable cone with an electrospray voltage less than 1000 V. Nozzles may be as small as 15 microns in diameter (Gary SchultzCornell University, http://www.cnf.cornell.edu/). The electrospray device may be interfaced to a time-of-flight mass spectrometer using continuous infusion of test compounds at the flow of rates less than 100 nL/min. Using such a system, a stable nanoelectrospray from a 20 micron diameter nozzle at 700 V and 100 n L/min of reserpine solution at 500 ng/ml in 50% water/50% methanol solution can be generated (Gary SchultzCornell University, http://www.cnf.cornell.edu/). For example, electrospray device lifetimes achieved thus far have exceeded 1 hr of continuous operation, a level which is sufficient for typical chip-based separations. Total volumes of less than 100 pL electrospray can be employed, a level which is suitable for combination with microfluidic separation devices.

The performance of this electrospray device is equivalent to conventional nanoelectrospray (nL electrospray) using a tapered fused-silica capillary. The electrospray device may be positioned up to 10 mm from the orifice of a TOF-MS to establish a stable nanoelectrospray. FIG. 4, shows a sketch of an electrospray device used for the arrays of the present invention. For example, a mass spectrum generated from the infusion of 1 mg/mL reserpine solution demonstrates a signal to noise ration of greater than 100, using a microchip-based electrospray device (Gary SchultzCornell University, http://www.cnf.cornell.edu/)

The use of multi-dimensional spectroscopy offers advantages over time-of-flight mass spectrometry and ion mobility instrumentation independently. The ability to rapidly assess isomer content provides a new approach to combinatorial analysis and screening. Integration software will be used to assess mass, charge, mobility and overall composition data on molecules in a mixture from a MDS instrument, and to create associated libraries for compounds assessed for their interaction with the array.

In another embodiment, components present on the arrays of the invention are assayed using collision induced dissociation (CID). CID occurs as an ion/neutral process wherein a (fast) projectile ion is dissociated as a result of interaction with a target neutral species. This is brought about by converting part of the translational energy of the ion to internal energy in the ion during the collision. By using the mobility of a parent ion as a label, fragments are assigned to parent ions after the CID process and sequence components in the mixtures in parallel. The key to providing a detailed large-scale mixture analysis is to identify sequence components in parallel. Our method should significantly improve the analysis of complex mixtures encountered during mixing and splitting synthetic processes used to generate combinatorial libraries as well as identification of peptides and proteins encountered in the emerging field of proteonics. Because of the ability to label and track both the parent and fragment molecules, CID is among the most powerful delineators of small ion structure and has recently emerged as a means of rapidly sequencing peptides and proteins (Hoaglund-Hyzer et al., Anal. Chem. 72, 2737-40, 2000).

### EXAMPLE 1

Isolation and Characterization of Sequences Used to Generate Expression System Arrays

A protein expression library can be created using mRNA, cDNA, or PCR amplified sequences of interest. CDNA libraries may be generated from random tissue samples, or may be generated from a tissue sample comprising a specific biological state, such as a tumor or specific organ. In addition, cDNA isolated from specific diseased tissue, or comprising a specific set of known ESTs (expressed sequence tags), is commercially available. For example, cDNAs from cancer cells or disease related cells are synthesized from mRNA by reverse transcriptase-polymerase chain reaction (RT-PCR) using reverse transcriptase with oligo (dT) or random hexametric oligonucleotides which have a restriction enzyme size for first strand synthesis, and a high fidelity DNA polymerase such as turbo pfu DNA polymerase from (Promega, Madison Wis.), platinum pfX DNA Polymerase (Life Technologies; Rockville, Mass.), or Advantage-HF 2 from (Clontech; Palo Alto, Calif.) for amplification of the cDNA.

To generate a library of related protein fragments, open reading frames of known protein targets identified in DNA databases are amplified by the polymerase chain reaction (PCR) for subcloning. For example, a receptor protein, enzyme binding domain, or enzyme catalytic site can be analyzed by computerized analysis for aspects of protein structure or function that are of interest. Programs used for proteomics analysis are well known in the art and include GCG (Genetics Computer Group; Madison, Wis.) and BLAST (see e.g http://www.ncbi.nlm.nih.gov), Pfam-HMM, ScanProsite, SMART, CD-Search, SIM (see e.g. http://www.ExPASy), and PeptideSearch (EMBL, Protein and Peptide Group). Proteins may be related based upon three dimensional structure analysis, amino acid analysis, functional domain, or upon known similarities of function. Also, proteins of the same family or from the same species may be used to generate the library. Once sequences of interest are identified, primers which flank those sequences are synthesized and the intervening sequences amplified by RT-PCR.

### EXAMPLE 2

Expression of Peptide/Protein Sequences

For most applications, in vivo expression of proteins is employed. Thus, cDNAs or PCR products are cloned into a commercial expression vectors such as LRCX retroviral vector set (Retro-X system; Clontech, Palo Alto, Calif.), MSCV retroviral expression system (Clontech; Palo Alto, CA), a baculovirus expression system (pFastBac; Life Technologies), or mammalian expression vectors which provide epitope tagging (e.g. pHM6 or pVM6, Roche Molecular Biochemicals, Indianapolis, IN; pFLAG, Sigma, St. Louis, Mo.).

Proteins can be expressed in an E. coli bacterial expression system using a plasmid vector or phage display vector. Bacterial expression systems are easy to manipulate and grow quickly. As discussed below, recombinant proteins can be expressed as a fusion protein with a specific “tag” sequence and proteolytic site that can help to purify or couple on to the arrays and cleave to remove the carrier after protein be purified.

Mammalian cells are often used as hosts for the expression of the cDNA that from higher eukaryotes because the signals for synthesis, processing, and secretion of these proteins are usually recognized. Cells may be transiently transfected, or stably transformed (by integration of the recombinant DNA into the host genome) depending on the requirements of the expression system. Generally, cloned cDNA is transiently transfected into the mammalian cell lines, such as COS cells, CVI, NIH 3T3, or Hep G2 cells. Transient transfection provides high-levels of expression (>10⁵ copies of plasmid DNA/cell), with host cells that are easy to manipulate. Expression is transient, however, because replication of the transfected plasmid continues unchecked until the cells die. Transient transfection in COS cells is the most widely used of all eukaryotic transfection systems.

The cDNA also can be used to generate stable transformants by transfecting mammalian cell lines, such as SK-Hep 1, C127, CHO. Stable transfection is performed by co-transfecting cells with DNA encoding a drug-resistance gene and the DNA of interest. Stable transfection is maintained by selecting for cells having drug resistance (e.g. G418, hygromycin, puromycin). Generally, stable transfection requires several months of cell passage and selection. However, once transformed, the cells grow continuously and express protein for several generations.

Retroviral systems are also widely used for expression of recombinant proteins. Retroviral vectors typically infect any mitotically active cell from a wide host range with nearly 100% efficiency. Generally, the target gene is cloned into the retroviral vector of choice. Once the packaging cells (containing viral DNA required for viral functions not encoded by the vector) are prepared, the vector/insert is transfected into the host cells. Recombinant virus (containing vector/insert and viral genome) is then used for large scale infection.

Recombinant DNA (i.e. vector plus insert) can be transformed or transfected into host cells using methods known in the art, such as electroporation or calcium phosphate-mediated precipitation. In general, the method used for transformation may depend on the host cell. Thus, ligated plasmid DNA can be transformed into cells made competent by treatment with calcium phosphate or electroporation (see e.g., Short Protocols in Molecular Biology, 2^{nd} Edition, Ausubel F. M .et al. 1992; Current Protocols: Molecular Cloning, Joseph Sambrook and David W. Russell, Cold Spring Harbor Laboratory Press, 2000). Calcium phosphate transfection is a widely used method for transfection. The transfected DNA enters the cytoplasm of the cell by endocytosis and is transferred to the nucleus. Depending on the cell type, up to 20% of a population of cultured cells can be transfected. Electroporation is also commonly used for transfection. In electroporation, the application of brief, high-voltage electric pulses to the host cell (mammalian and/or plant) cells leads to the formation of small (nanometer sized) pores in the plasma membrane. DNA is taken directly into the cell cytoplasm. Finally, liposomes are also used for transfection of mammalian cells. In liposome-mediated transfection, artificial membrane vesicles (liposomes) which include encapsulated of DNA or RNA are fused with the cell membrane.

### EXAMPLE 3

Assay of Recombinant Proteins Expressed in vivo as an Array

Host cells comprising recombinant proteins/peptides (i.e. host cells transfected with sequences encoding protein/peptides inserted into an expression vector suitable for the host) are incubated at 37° C. overnight, and single colonies or plaques picked for immobilization on the array. After transfection, cells are put into the array wells and incubated at 37° C. for 6-8 hr. The cells attach on the on bottom of the array wells and can be used for detecting expressed proteins of interest.

For example, in an embodiment, the expressed proteins comprise membrane anchoring sequences and are localized on the cell surface (FIG. 3C). With the expression systems placed in such an array, small molecules, peptides, proteins, or other compounds of interest in solution or libraries of said compounds may be exposed to the array. After incubation with the array, any non-binding compounds can be washed away and binding interaction with the various proteins detected by various analytical methods such as ELISA, receptor binding assays and high throughput spectroscopy such as MDS and the like.

Secreted proteins can also be assayed in situ (FIG. 3A, bottom), or can be transferred into a separate array (FIG. 3A, top). Recombinant proteins which include a tag, such as poly-histidine may be immobilized in the well by coating wells with a layer of metal ions. Thus, the present invention contemplates that arrays are generated with metal ion as part of the binding surface for immobilization of secreted proteins. Alternatively, tagged secreted proteins can be transferred into a separate array (FIG. 3B, top) made with metal ion as part of, or coated onto, the binding surface (FIG. 3B, top).

For example, by including the sequence encoding specific residues, expressed proteins can be synthesized with a tag, such as His₆ (six histidine residue epitope) by including the sequence (CAC)₆ in the primer used for PCR or by using a vector which includes the tag (e.g. pM6 or pVM6 epitope tagging vector; Roche Molecular Biologicals). Polyhistidine-tagged fusion proteins can be purified with TALON metal affinity resin (Clontech). Other tagging vectors which are commercially available include tags recognized by antibodies to the peptide tag. Antibody-binding tags include peptides derived from the human c-myc protein (nine amino acid epitope), Protein-C (a twelve amino acid epitope from the heavy chain of human Protein-C), Hemagglutinin (HA), FLAG (8 amino acid), and the like.

In some applications, it is necessary to remove the tag. To provide for easy removal of the tag, expressed proteins may be generated to include protease-sensitive cleavage site such as thrombin recognition sequence (P4-P3-Pro-Arg (or Lys)•P1′-P2′; P2-Arg (or Lys)P1′ or enterokinase recognition sequence (Asp₄-Lys•X) adjacent to the tag. Protease sites may be engineered into a vector by PCR-based oligonucleotide mutagenesis, or added to the inserts by synthesizing primer with the sequence.

### EXAMPLE 4

Assay of Recombinant Receptor for Advanced Glycation End Products (RAGE) Produced by an Array of Protein Expression Systems.

NIH 3T3 or 293 cells were grown to about 80% confluence in 60 mm dishes using DMEM or EMEM with 10% fetal calf serum, respectively. The cells were transfected with RAGE-pCDNA, a recombinant plasmid having an insert encoding sequences derived from the Receptor for Advanced Glycation End Products (RAGE). Transfections were performed using 2 μg/well DNA and 6 μl FuGENE 6 (Roche Molecular Biochemicals, Indianapolis, IN). At 40 h post-transfection, cells were detached by treatment with 0.05% trypsin and 0.53 mM EDTA, and transferred into 96 well or 384 well microtiter, and incubated for 4-8 h to allow the cells to attach to the bottom of the well. The array (comprising RAGE-expression vector system in the cells) is then frozen with 5% (v/v) DMSO-medium or fixed with 4% (v/v) formaldehyde for long-term storage.

The array or plate was washed with phosphate buffered saline, pH 7.2 (PBS) or medium, blocked with 1% BSA in PBS for 1 h at room temperature, and then incubated with a RAGE ligand such as S100b, CML or P-amyloid with or without compound for 1 h at 37° C. The arrays were washed six times with 0.05% Tween 20 in 10 mM Tris-HCl, 150 mM NaCl, pH 7.2. The ligand and receptor binding were detected with anti-ligand secondary antibody conjugated with alkaline phosphatase. The alkaline phosphatase substrate solution (p-nitrophenylphosphate in 1 M diethanolamine, pH 9.8) was added into the array and developed for 30-60 min at room temperature in the dark, and after the addition of stop solution (5% EDTA) the absorbance at 405 nm measured.

Alternately, binding assays may be performed using ¹²⁵I-ligand, fluorescent-labeled ligand and the like. For example, ¹²⁵I radioactivity bound to the expressed receptor can be measured using a Gamma counting system or detected by autoradiography. The fluorescent conjugate can be detected by fluorescence microscopy or confocal microscopy. In other applications, compounds that inhibit receptor ligand binding are evaluated by measuring the ability of the compound of interest to inhibit binding of the known ligand.

Thus, the present invention provides a means of rapid characterization of compound-protein interaction. In addition, the present invention provides a means to characterize small molecule libraries, protein or peptide libraries, or single compounds against an array of proteins in a single experiment, generate information about the protein structure, and sequence and reexpress the protein or proteins of interest make this an extremely powerful tool for the pharmaceutical, agrochemical and environmental industry.

With respect to the descriptions set forth above, optimum dimensional relationship of parts of the invention (to include variations in specific components and manner of use) are deemed readily apparent and obvious to those skilled in the art, and all equivalent relationships to those illustrated in the drawings and described in the specification are intended to be encompassed herein. The foregoing is considered as illustrative only of the principal of the invention. Since numerous modifications and changes will readily occur to those skilled in the art, it is not intended to limit the invention to the exact embodiments shown and described, and all suitable modifications and equivalents falling within the scope of the appended claims are deemed within the present inventive concept.

It is to be further understood that the phraseology and terminology employed herein are for the purpose of description and are not to be regarded as limiting. Those skilled in the art will appreciate that the conception on which this disclosure is based may readily be used art as a basis for designing the methods and systems for carrying out the several purposes of the present invention. The claims are regarded as including such equivalent constructions so long as they do not depart from the spirit and scope of the present invention. All patents and publications cited herein are fully incorporated by reference in their entirety.

What is claimed is:

1. A spatially defined array of protein expression systems comprising
(a) a substrate;
(b) a binding surface which covers some or all of the substrate surface; and
(c) a plurality of protein expression systems located at discrete positions on portions of said substrate covered by said binding surface.

2. The array of claim 1, wherein said expression systems produce recombinant proteins.

3. The array of claim 2, wherein said proteins produced by said expression systems are immobilized on said array.

4. The array of claim 3, wherein said immobilization of said proteins produced by said expression systems comprises immobilization of said expression systems.

5. The array of claim 3, wherein said immobilization of said proteins produced by said expression systems comprises a direct interaction of said expressed protein with said binding sur face.

6. The array of claim 2, wherein said expressed proteins comprise an affinity tag.

7. The array of claim 2, wherein the expressed proteins comprise an epitope tag.

8. The array of claim 1, wherein each discrete position on the array comprises one protein expression system.

9. The array of claim 1, wherein each discrete position on the array comprises a plurality of protein expression system.

10. The array of claim 1, wherein each protein expression system expresses a unique protein or peptide.

11. The array of claim 1, wherein at least some of the expression systems express peptides or protein fragments derived from the same protein.

12. The array of claim 1, wherein at least some of the expression systems express related .proteins.

13. The array of claim 12, wherein said related proteins are related functionally.

14. The array of claim 12, wherein said related proteins are related structurally.

15. The array of claim 1, wherein at least a subset of the proteins expressed by the protein expression systems of the array are members of the same family.

16. The array of claim 15, wherein said family of proteins expressed by the protein systems of the array comprises growth factor receptors, hormone receptors, neurotransmitter receptors, catecholamine receptors, amino acid derivative receptors, cytokine receptors, extracellular matrix receptors, antibodies, lectins, cytokines, serpins, proteinases, kinases, phosphatases, ras-like GTPases, hydrolases, steroid hormone receptors, transcription factors, DNA binding proteins, zinc finger proteins, leucine-zipper proteins, homeodomain proteins, intracellular signal transduction modulators and effectors, apoptosis-related factors, DNA synthesis factors, DNA repair factors, DNA recombination factors, cell-surface antigens, Hepatitis C virus (HCV) proteases, HIC proteases, viral integrases, or proteins from pathogenic bacteria.

17. The array of claim 1, further comprising at least 10 discrete locations comprising protein expression systems on one array.

18. The array of claim 1, further comprising at least 102 discrete locations comprising protein expression systems on one array.

19. The array of claim 1, further comprising at least 103 discrete locations comprising protein expression systems on one array.

20. The array of claim 1, further comprising at least 10⁴ discrete locations comprising protein expression systems on one array.

21. The array of claim 1, wherein said binding surface comprises a component which binds to said protein expression systems.

22. The array of claim 21, wherein the binding surface comprises an antibody which binds to said protein expression systems.

23. The array of claim 1, wherein said binding surface comprises a hydrogel.

24. The array of claim 1, wherein said binding surface comprises a membrane.

25. The array of claim 1, wherein said binding surface comprises at least one functional group that binds to the substrate and at least one functional group that binds to said protein expression systems.

26. The array of claim 1, wherein the binding surface comprises a compound which binds to the proteins produced by said protein expression systems.

27. The array of claim 26, wherein said binding surface comprises an antibody which binds to the proteins produced by said protein expression systems.

28. The array of claim 26, wherein said binding surface comprises at least one layer of coating material.

29. The array of claim 26, wherein said coating comprises a metal film.

30. The array of claim 1, wherein the substrate is selected from the group consisting of silicon, silicon dioxide, alumina, glass, titania, nylon, polypropylene, polyethylene, polystyrene, and acrylamide.

31. A micromachined device comprising the array of protein expression systems of claim 1.

32. A biosensor comprising the array of protein expression systems of claim 1.

33. A method for screening a plurality of proteins for their ability to interact with a component of a sample comprising the steps of:
(a) generating a protein expression array, wherein the array comprises:
(i) a substrate;
(ii) a binding surface which covers some or all of the substrate surface; and
(iii) a plurality of protein expression systems located at discrete positions on portions of the substrate covered by the binding surface; and
(b) detecting either directly or indirectly the interaction of the component with proteins expressed at specific positions comprising the protein expression systems.

34. The method of claim 33, wherein the method comprises detecting the component retained at a specific position on the expression array.

35. The method of claim 33, wherein the method comprises transferring the expressed proteins to known locations on a second array and detecting the interaction of the components of the sample with the second array.

36. The method of claim 33, wherein the step of detection comprises characterization of binding of the components to proteins expressed from protein expression systems located at specific positions on the array.

37. The method of claim 33, wherein the step of detection comprises characterization of an alteration in the activity of proteins expressed from protein expression systems located at specific positions on the array.

38. The method of claim 33, further comprising characterization of DNA isolated from the expression system for which the interaction is detected.

39. The method of claim 33, wherein the component tested for interaction with the proteins expressed by the protein expression systems of the array comprises a protein or peptide.

40. The method of claim 33, wherein the component tested for interaction with the proteins expressed by the protein expression systems of the array comprises a small molecule.

41. The method of claim 33, wherein the component tested for interaction with the proteins expressed by the protein expression systems of the array comprises a proprotein.

42. The method of claim 33, wherein the component tested for interaction with the proteins expressed by the protein expression systems of the array comprises a receptor ligand.

43. The method of claim 42, wherein the ligand is selected from the group consisting of peptides, peptide mimetics, antibodies, small molecules, natural product extracts, and mixtures of the above.

44. The method of claim 33, wherein the interaction of the components of a sample with the expression array is measured by multi-dimensional spectroscopy utilizing ion mobility and time of flight mass spectroscopy for the detection of biological or chemical products formed as the result of the interaction of at least one component of the sample with proteins expressed from specific sites on the protein expression array.

45. The method of claim 44, comprising the steps of:
(a) recovering at least a portion of said biological or chemical products formed as the result of the interaction of components of a sample with proteins expressed from specific sites on the protein expression array as an electrospray;
(b) directing the electrospray to an ion mobility chamber which separates the constituents of the directed electrospray based on size, ionic charge, and shape; and
(c) analyzing the separated constituents of the directed electrospray which emerge from the ion chamber by time-of-flight spectroscopy.

46. The method of claim 33, wherein the interaction of the components of a sample with the expression array is measured by multi-dimensional spectroscopy utilizing ion mobility and time of flight mass spectroscopy for the detection of biological or chemical products formed as the result of the interaction of at least one component of the sample with proteins expressed from specific sites on the protein expression array.

47. A method for the detection of chemical or biological components immobilized on a solid phase by multidimensional spectroscopy (MDS) utilizing ion mobility and time of flight mass spectroscopy comprising the steps of:
(a) recovering at least a portion of a chemical or biological mixture immobilized on a solid substrate as an electrospray;
(b) directing the electrospray to an ion mobility chamber which separates the constituents of the directed electrospray by size, ionic charge, and shape; and
(c) analyzing the separated constituents which emerge from the ion chamber by time-of-flight spectroscopy.

48. The method of claim 47, wherein the immobilized components are immobilized as an array.

49. The method of claim 47, wherein the array comprises a microchip format.

50. The method of claim 47, wherein the array comprises an array of protein expression systems or products thereof.

51. Computer readable media comprising software code for performing the method of claim 47.
